(19) United States
(12) Patent Application Publication
(10) Pub. No.: US 2019/0125216 A1
(43) Pub. Date: May 2, 2019

(54) Gait Monitoring and Stimulation Device

(71) Applicant: Crystal Salcido, El Paso, TX (US)

(72) Inventor: Crystal Salcido, El Paso, TX (US)

(21) Appl. No.: 16/177,572

(22) Filed: Nov. 1, 2018

Related U.S. Application Data
(60) Provisional application No. 62/580,092, filed on Nov. 1, 2017.

(51) Int. Cl. A61B 5/11; A61B 5/00

(52) U.S. Cl. CPC A61B 5/112; A61B 5/1114; A61B 5/4023; A61B 5/1118; A61H 3/00; A61B 5/1104; A61B 5/742; A61B 5/7282; A61B 5/4082; A61B 2562/0219

(57) ABSTRACT

A gait monitoring and stimulation device utilizing multiple sensors for actively monitoring for gait freezing events and utilizing multiple types of gait stimulation cues or modes in response to the detection of a gait freezing event experienced by a user. The gait monitoring and stimulation device utilizes global positioning system geographic data, accelerometer data, and audio data to determine whether a gait freezing event is likely being experienced by a user. A laser device used in conjunction with a lens having a diffractive optical element, is utilized to project a visual cue comprising the image of a descending staircase onto the ground in front of the user of the device. Auditory stimulation cues are also provided. One or more accelerometers monitor the orientation of the device, deactivating the laser if the device orientation exceeds a threshold angle likely to lead to eye damage caused by the laser.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Technical Field

The present invention relates to electronic devices for providing visual and auditory stimulation to users and, more specifically, to a gait monitoring and stimulation device for actively monitoring for multiple indicators of gait irregularities that may indicate gait freezing by persons suffering from Parkinson's disease and other neurological disorders, and safely providing a plurality of types of gait visual, audio, and/or vibratory-based stimulation cues.

### Description of Related Art

Persons suffering from Parkinson's disease and other neurological disorders often experience gait irregularities. The most severe form of such gait irregularities occurs when a person experiences a gait “freezing” event, which results in the person exhibiting cessation of all walking movements. Such freezing events can occur without warning and may occur multiple times during the course of a short walk. Freezing events can substantially impede a person's mobility and may also pose safety hazards to the person when, for example, such a person experiences a freezing event while walking across a street. Freezing events can also cause a person to experience feelings of embarrassment, often leading the person to become more sedentary.

Studies have revealed that providing visual cues to a person experiencing such gait freezing events may alleviate or decrease the duration of such events, or prevent them from occurring altogether. Light sources, including lasers, may be used to provide visual cues to such persons during or before such freezing events. For example, a light source such as a laser may be utilized to project a series of dots on the ground in front of a person suffering from Parkinson's disease. While the precise reasons why such visual cues are successful in alleviating or preventing freezing events is the subject of much research and speculation. However, the result of presentation of the visual cues is that it often encourages the person to begin taking additional steps and to feel more relaxed and confident. Auditory cues presented to a person suffering from Parkinson's disease, such as the sound of a metronome or other pulse or continuous sounds, may also be utilized to alleviate freezing events or to prevent them from occurring.

Prior art devices used to monitor gait freezing events and to provide gait stimulation cues have several drawbacks. One drawback is that such prior art devices do not provide for multiple means by which to monitor for gait freezing events, including multiple sensory data associated with both geographic location and distance traveled, movement, and sound volume surrounding the device. Another drawback is that such prior art devices do not provide for the presentation of multiple types of gait stimulation cues, nor for the ability of a user or physician to quickly select such types of cues for use in a device. Another drawback of the prior art devices is that such devices do not provide for multiple means for activating one or more gait stimulation cues or modes. Another drawback of such prior art devices is that they do not provide means for deactivating certain visual cues, especially laser cues, when the device is oriented in a position that could potentially cause a laser beam to shine in the eyes of a user or adjacent person, possibly resulting in eye damage.

Therefore, what is needed is a gait monitoring and stimulation device that is capable of utilizing multiple types of sensor data to provide for gait monitoring. What is also needed is a gait monitoring and stimulation device that is capable of presenting multiple types of gait stimulation cues or “modes.” What is also needed is a gait monitoring and stimulation device that may be activated to provide gait stimulation cues in multiple ways, including via verbal commands. What is also needed is a gait monitoring and stimulation device that includes means for deactivating laser devices if the gait monitoring and stimulation device is oriented in such a manner that the laser beam could potentially cause eye damage to a user or adjacent person. The gait monitoring and stimulation device described herein satisfies these needs and others as will become apparent to one of ordinary skill after a careful study of the detailed description of the embodiments set forth below.

### BRIEF DESCRIPTION OF THE SEVERAL VIEWS OF THE DRAWINGS

The present invention will be more fully understood by reference to the following detailed description of the preferred embodiments of the present invention when read in conjunction with the accompanying drawings, wherein:

FIG. 1 is a perspective view of an embodiment of the gait monitoring and stimulation device;

FIG. 2 is a side cross-sectional view of the embodiment of the gait monitoring and stimulation device appearing in FIG. 1;

FIG. 3 is a front cross-sectional view of the embodiment of the gait monitoring and stimulation device appearing in FIG. 1;

FIG. 4 is a partial exploded view of a forward portion of a laser device body, lens, and laser device tip of an embodiment of a laser device mounted within the embodiment of the gait monitoring and stimulation device appearing in FIG. 1;

FIG. 5 is a side cross-sectional view of the forward portion of the embodiment of the laser device body, lens, and laser device tip appearing in FIG. 4;

FIG. 6 is a side cross-sectional view of a forward portion of an alternate embodiment of a laser device body, lens, and laser device tip that may be utilized in an alternate embodiment of a gait monitoring and stimulation device;

FIG. 7 illustrates a side view of a user of an embodiment of a gait monitoring and stimulation device which is projecting, via a laser, an image of a descending staircase in front of the user;

FIG. 8 illustrates a top view of the descending staircase image appearing in FIG. 7;

FIG. 9 illustrates an embodiment of a lens having a diffractive optical element for use in connection with a gait monitoring and stimulation device configured to display, via a laser device, a descending staircase image of the type appearing in FIG. 8;

FIG. 10 illustrates a block diagram showing exemplary components of an alternate embodiment of a gait monitoring and stimulation device;

FIG. 11 is a process flow diagram illustrating steps of monitoring for gait irregularities and providing gait stimulation performed by an embodiment of the gait monitoring and stimulation device; and

FIG. 12 is a process flow diagram illustrating steps for the manual activation of selected gait stimulation modes performed by an embodiment of the gait monitoring and stimulation device.

The above figures are provided for the purpose of illustration and description only, and are not intended to define the limits of the disclosed invention. Use of the same reference number in multiple figures is intended to designate the same or similar parts. Furthermore, if and when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the particular embodiment. The extension of the figures with respect to number, position, relationship, and dimensions of the parts to form the preferred embodiment will be explained or will be within the skill of the art after the following teachings of the present invention have been read and understood.

### DETAILED DESCRIPTION OF THE INVENTION

Several exemplary embodiments of the claimed invention(s) will now be described with reference to the drawings. Unless otherwise noted, like elements will be identified by identical numbers throughout all figures. The invention(s) illustratively disclosed herein suitably may be practiced in the absence of any element that is not specifically disclosed herein.

Systems and methods for monitoring for gait freezing events and for safely providing gait stimulation cues via a device are disclosed herein. It should be noted that while the exemplary embodiments described herein are associated with gait freezing events often experienced by persons with Parkinson's disease, the devices and methods taught below could also be equally utilized in connection with other types of neurological disorders that produce various types of gait irregularities or other types of movement related irregularities that benefit from the providing of stimulation cues. It should also be noted that although the gait monitoring and stimulation device has been described herein in the context of a handheld embodiment of the device, other alternate embodiments of the device may be worn by a user, or may be mounted to objects such as walkers, walking canes, and any other object capable of holding the device.

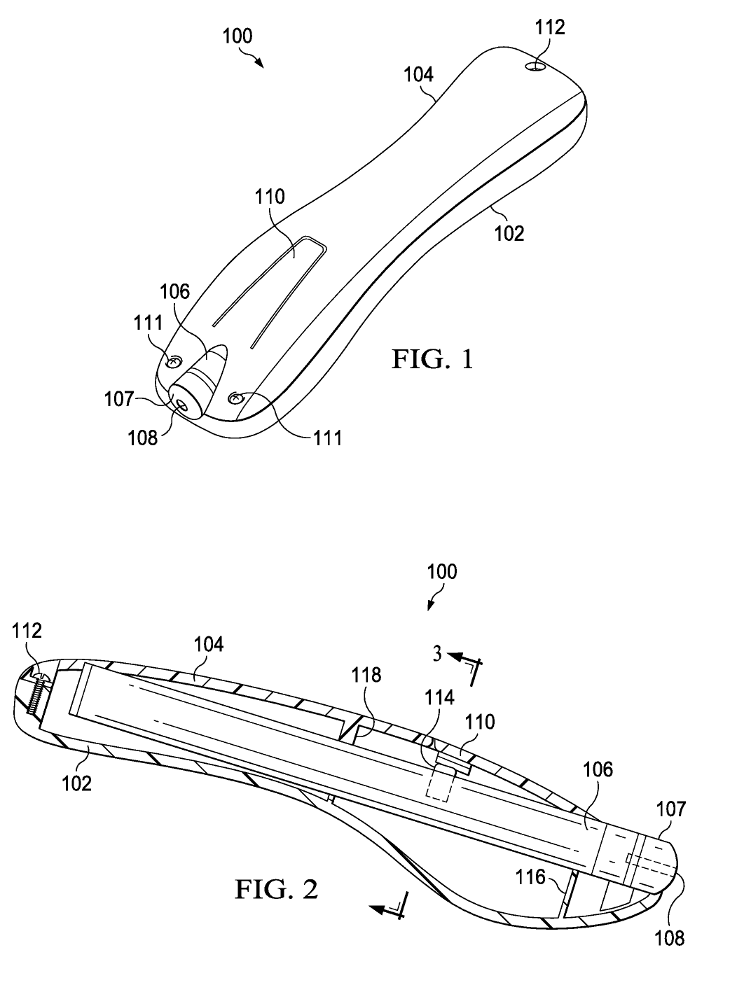

Referring now to FIG. 1, a perspective view of an embodiment of the gait monitoring and stimulation device 100 is shown. In one embodiment of the gait monitoring and stimulation device 100, the device includes an upper housing 104 and a lower housing 102 secured together by a plurality of fasteners (111 at the anterior end of the housing, 112 at the posterior end of the housing). The housing has a anterior end and a posterior end. In one embodiment of the gait monitoring and stimulation device, a laser device 106 is mounted between the upper housing 104 and lower housing 102 such that only an anterior or “forward” portion of the laser device 106 is visible and protrudes through an opening in the anterior end of the housing. In one embodiment, a flexible tab 110, a cut-out portion of the upper housing, is integrated into the upper housing of the gait monitoring and stimulation device such that a user may depress such tab to activate the laser device 106 mounted within the upper and lower housing of the gait monitoring and stimulation device. An anterior end of the laser device includes a laser tip body which, on an anterior end, has an aperture through which a laser beam passes and, on posterior end (not shown at FIG. 1), has threads formed to allow the laser tip body to mate with a correspondingly threaded recess formed on the forward portion of the laser device.

Referring now to FIG. 2, a side cross-sectional view of the embodiment of the gait monitoring and stimulation device 100 appearing in FIG. 1 is shown. Laser device mounting structures (116, 118) are formed within the interior portions of the upper housing 104 and lower housing 102, said laser device mounting structures configured to stabilize the laser device 106 within the gait monitoring and stimulation device such that movement is eliminated or minimized. In one embodiment, one end of the aforesaid flexible tab 110 is positioned to be adjacent to a power button 114 located on an upper side of the laser device 106 such that by depressing the flexible tab 110, a user may in turn depress the power button 114 of the laser device, thus allowing the laser device to be easily turned on and off by a user.

In one embodiment, the upper and lower housing of the gait monitoring and stimulation device provides a structure that may be easily held by a user. As shown in FIG. 2, a lower portion of the gait monitoring and stimulation device may be curved such that an anterior portion of the device is thicker than a posterior portion of the device, which may allow for an elderly user to more easily grasp the device, and may allow for a more natural downward orientation of the device. The housing of the gait monitoring and stimulation device may also assist in preventing unwanted debris or other unwanted elements from entering the housing or easily coming into contact with any device component but the laser tip body. It is contemplated that in some alternate embodiments of the gait monitoring and stimulation device, the housing will provide a watertight enclosure, not exposing any portion of the laser device. Such a watertight enclosure, which may include a transparent window through which the laser beam may pass, may be advantageous when using the gait monitoring and stimulation device in adverse weather conditions.

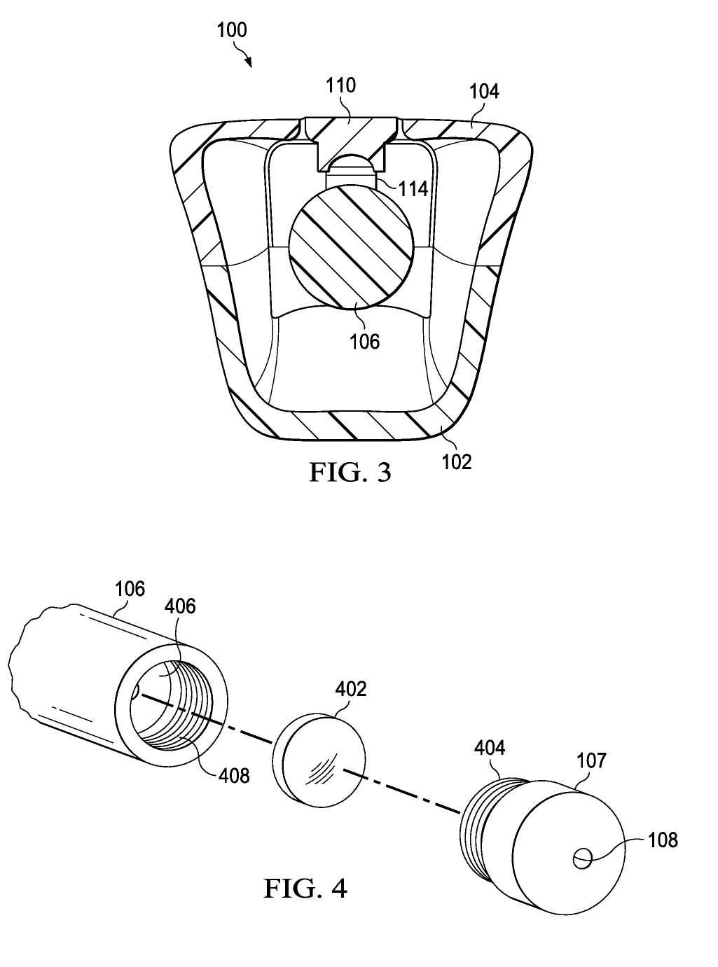

Referring now to FIG. 3, a front cross-sectional view of the embodiment of the gait monitoring and stimulation device appearing in FIG. 1 is shown. In one embodiment, the flexible tab 110 formed on the upper housing 104 of the gait monitoring and stimulation device has a curved lower surface configured to engage a correspondingly curved power button 114 positioned on the top side of the laser device 106. Accordingly, the rounded lower surface of the flexible tab 110 not only acts as a structure by which a user may activate the laser device to power it on or off, but such flexible tab also acts as a structure by which the laser device is further stabilized within the upper and lower housing of the gait monitoring and stimulation device to eliminate or substantially reduce movement of the laser device within the housing.

In one embodiment, the gait monitoring and stimulation device housing is constructed of a rigid plastic material, but in other alternate embodiments, the device may be constructed of other materials such as metal or carbon fiber. In alternate embodiments of the gait monitoring and stimulation device, the housing may include a hole through which a lanyard or other strap may be attached such that the lanyard or strap can be worn by a user to prevent the dropping of the device.

In one embodiment, the laser device 106 comprises an off-the-shelf laser pointer. For example, in one embodiment, a self-contained laser pointer made by Laser Points, having a 532 nanometer wavelength, with an output power of 5 milliwatts, powered by three AAA batteries, and emitting a green colored single line beam having a beam diameter of less than 2.5 millimeters, may be utilized in conjunction with a lens, not off-the-shelf, having diffractive optical elements as discussed below in connection with FIG. 9, to produce a visual gait stimulating cue for terminating or reducing the intensity or duration of gait freezing events of the type suffered by those with Parkinson's disease. Other alternate embodiments of the gait monitoring and stimulation device may utilize other types of laser devices capable of emitting different types and colors of beams.

It should be noted that while the gait monitoring and stimulation device shown in FIGS. 1-3 illustrates an embodiment of the device that includes a self-contained laser device within the housing and little else in the way of components, it is fully contemplated herein and further discussed below that alternate embodiments of the gait monitoring and stimulation device may include more complex components allowing not only for active monitoring for gait irregularities by receiving data from multiple types of sensors, but also for the provision of multiple modes of providing gait stimulation cues, including both visual cues, auditory cues, and vibratory cues.

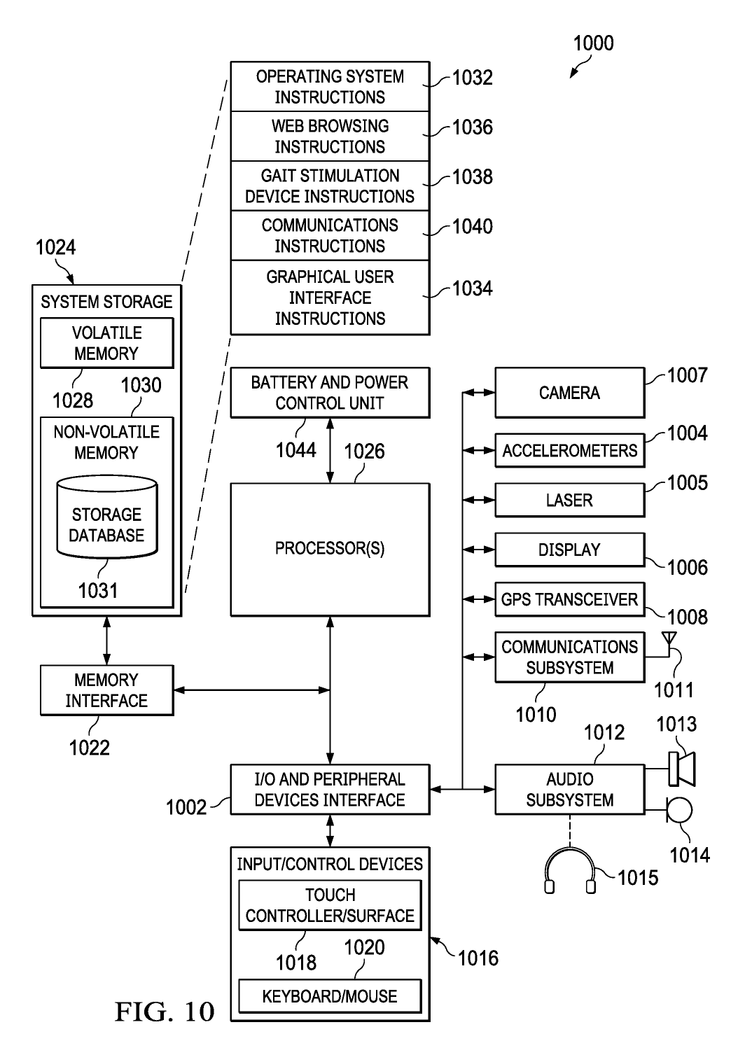

Likewise, as further discussed below, other alternate embodiments of the gait monitoring and stimulation device may include sensors (accelerometers or gyroscopes in communication with the processor) utilized for monitoring the orientation or tilt of the gait monitoring and stimulation device, so as to provide for deactivation of the laser emitting portion of the device in the event that the device's orientation is such that eye damage from the laser is possible. The types of components that would allow for such functionality, such as, for example, those components illustrated in FIG. 10, may be mounted within and/or on the housing of alternate embodiments of the gait monitoring and stimulation device. In other alternate embodiments, the gait monitoring and stimulation device could be incorporated into, or be an attachment of, a mobile computing device such as a smartphone or personal data assistant. For example, a laser device may be integrated into a mobile computing device as described in U.S. Pat. No. 6,065,880, issued on May 23, 2000, incorporated by reference herein. Other aspects and components of an embodiment of a more complex gait monitoring and stimulation device are described in more detail below with reference to FIG. 10.

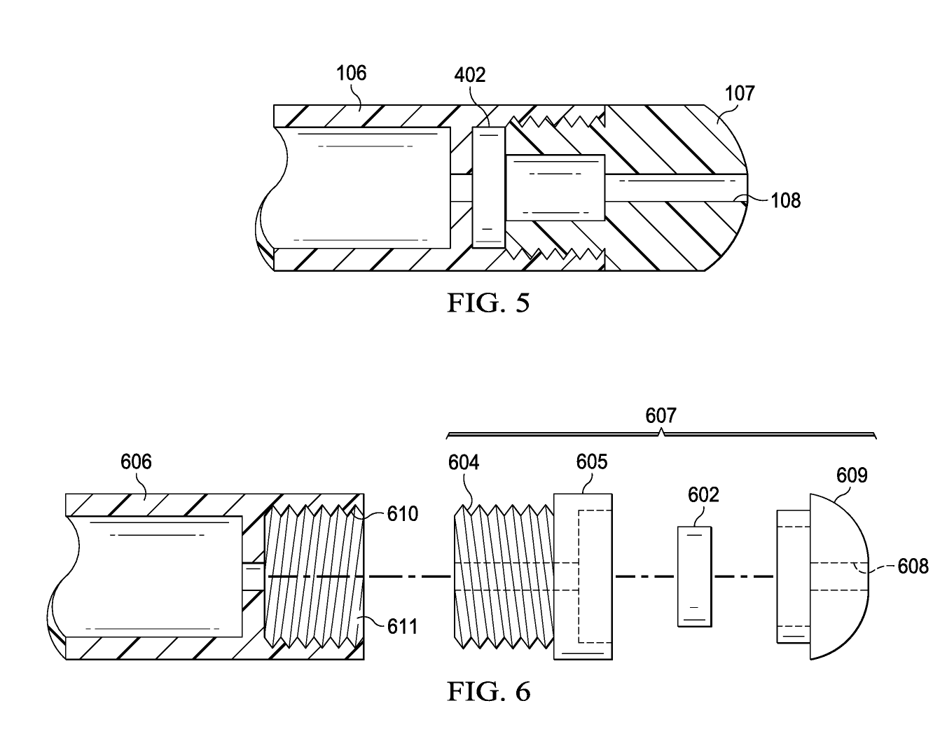

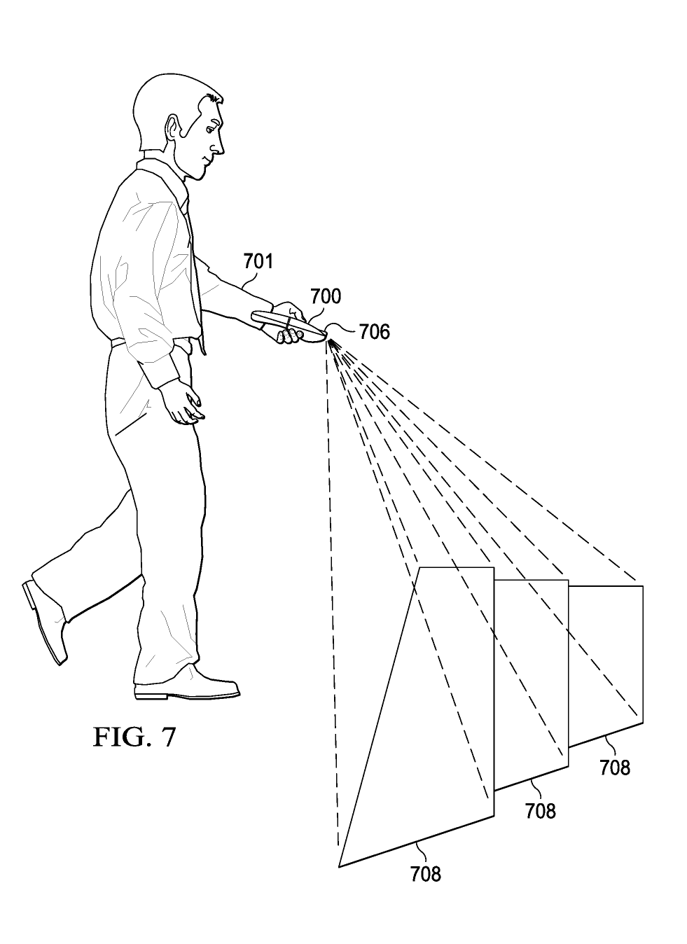

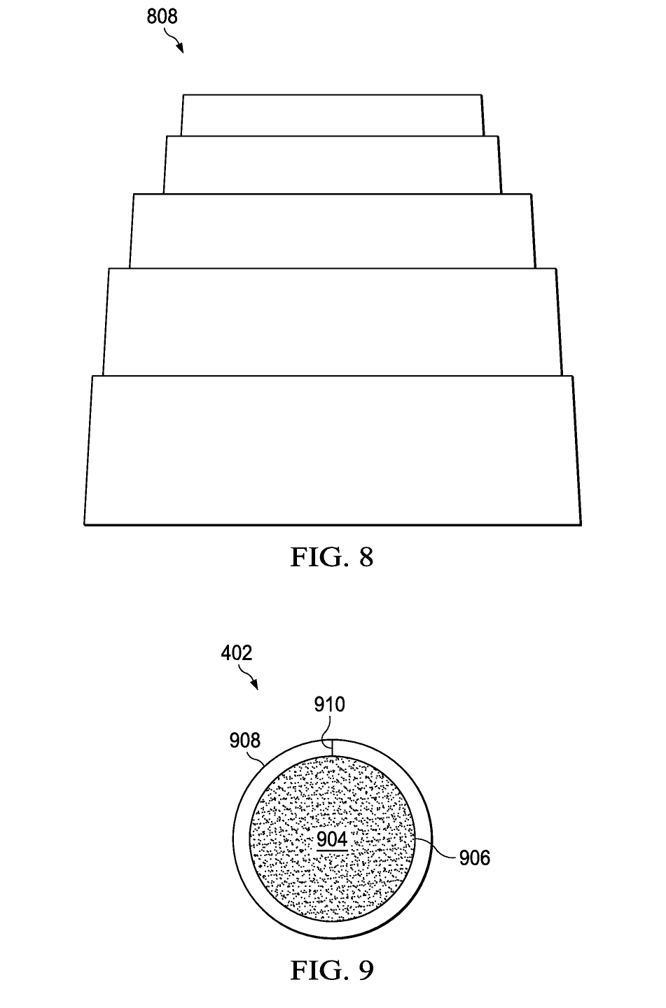

Referring now to FIG. 4, a partial exploded view of a forward portion of a laser device body 106, lens 402, and laser device tip 107 of an embodiment of a laser device mounted within the embodiment of the gait monitoring and stimulation device appearing in FIG. 1 is shown. An anterior end of the laser device tip 107 includes an aperture 108 through which a laser beam projecting a visual gait stimulating cue of the type appearing in FIGS. 7 and 8 may pass. A posterior end of the laser device tip, generally cylindrical in shape in the embodiment shown in FIG. 4, includes a cylindrical recess (not shown in FIG. 4) with curved interior walls as depicted in the cross-sectional view shown in FIG. 5, with a threaded exterior surface 404 configured to mate with a correspondingly threaded recess 406 of the laser device body 106 having threads 408 formed on the interior walls of such recess 406. A lens 402 having etched thereon a diffractive optical element, described in more detail below with reference to FIG. 9, is seated in such recess 406, between the laser body 106 and the laser tip 107. As described below, the lens having a diffractive optical element works to display the gait stimulating visual cue to eliminate or reduce the duration of freezing events of users.

Referring now to FIG. 5, a side cross-sectional view of the forward portion of the embodiment of the laser device body, lens, and laser device tip appearing in FIG. 4 is shown. In one embodiment as shown in FIG. 5, the lens having a diffractive optical element is mounted between the laser body 106 and the laser tip 107. In alternate embodiments of the gait monitoring and stimulation device, the device may provide for the interchangeability of the lens by a user. For example, some users may find that different types of visual cues provided by the laser device are effective, depending on the circumstances of use. In such alternate embodiments, the gait monitoring and stimulation device may provide the user with the ability to remove the laser tip body and lens, and insert a new lens providing for a different type of visual laser cue pattern. Such interchangeability of lens provides an advantage over prior art gait stimulation devices that provided only a single type of visual cue.

Referring now to FIG. 6, a side cross-sectional view of a forward portion of an alternate embodiment of a laser device body, lens, and laser device tip that may be utilized in an alternate embodiment of a gait monitoring and stimulation device is shown. In the embodiment shown in FIG. 6, the lens is mounted within the laser tip body. Specifically, an anterior end of a laser device tip cap 609 includes an aperture 608 through which a laser beam projecting a visual gait stimulating cue of the type appearing in FIGS. 7 and 8 may pass. A posterior end of the laser device tip, generally cylindrical in shape in the embodiment shown in FIG. 6, includes a cylindrical recess with curved interior walls, with a threaded exterior surface 604 configured to mate with a correspondingly threaded recess 610 of the laser device body 606 having threads 611 formed on the interior walls of such recess 610. A lens 602 having etched thereon, a diffractive optical element, is seated between the laser tip cap 609 and the posterior portion of the laser tip 605. A posterior end of the laser tip cap includes a recess shaped and sized to allow for the seating of the lens 602. A recess is also formed on the anterior end of the laser tip posterior element to receive the laser tip cap body as shown in FIG. 6. As described below, the lens 602 having a diffractive optical element works to display the gait stimulating visual cue to eliminate or reduce the duration of freezing events of users. Mounting of the lens within the laser tip as depicted in FIG. 6 allows for the interchangeability of lens by removing the laser tip and replacing it with another laser tip having a lens configured to project a different type of laser pattern for serving as a gait stimulating visual cue. Interchangeability of laser tips advantageously allows for changes of lens (and thus projected laser patterns) without users contacting lenses, which is desired when utilizing lenses with certain types of diffractive optical elements that may be damaged by human contact.

Referring now to FIG. 7, a side view of a user of an embodiment of a gait monitoring and stimulation device which is projecting, via a laser, a two-dimensional image of a descending staircase in front of the user. The gait monitoring and stimulation device 700 is configured to be held by a user 701 in a slightly downward orientation, allowing for the projection of a visual stimulating cue via, in one embodiment, a laser device 706. In one embodiment, one visual stimulating cue is a laser projected image of multiple adjoining trapezoid shaped images such that the trapezoid shape projected closest to the user has the greatest width, with each adjoining trapezoid, further away from the user, having a decreased width as depicted in FIG. 7. To a user, the adjoining plurality of trapezoids displayed by the laser device, in conjunction with a lens having a diffractive optical element, may appear as a descending staircase. Such an image of a descending staircase works as a gait stimulating cue to those with Parkinson's disease, allowing for the cessation of a gait freezing event. In alternate embodiments of the device, other types of two-dimensional images and three-dimensional images may be projected, via the diffractive optical element, to provide a visual stimulation cue.

Referring now to FIG. 8, a top view of the descending staircase image 808, comprising adjoining trapezoids (adjoined along the wider sides), appearing in FIG. 7 is shown. The image 808 projected acts as an illusion in that sense that it creates in the mind of the user of the gait monitoring and stimulation device, the sense of the user walking on a staircase, which often results in the user experiencing increased stabilization during walking, may prevent gait freezing events, and may cause for the cessation of gait freezing events should they occur. Users of the gait monitoring and stimulation device often experience feelings of relaxation and safety upon viewing the staircase image projected by the gait monitoring and stimulation device.

Referring now to FIG. 9, showing an embodiment of a lens having a diffractive optical element for use in connection with a gait monitoring and stimulation device configured to display, via a laser device, a descending staircase image of the type appearing in FIG. 8. A lens having a diffractive optical element (“DOE”) utilizes a surface with a complex microstructure for its optical function. The micro-structured surface relief profile has two or more surface levels. The surface structures can be replicated from a suitable tool by microembossing in various polymer materials. A laser beam projected through such a lens having a diffractive optical element may be configured to generate various 2D and holographic patterns. Examples of such diffractive optical elements are described in U.S. Pat. No. 5,938,308, issued on Aug. 17, 1999, incorporated by reference herein.

Still referring to FIG. 9, in one embodiment, the lens 402 of the gait monitoring and stimulation device is constructed of a optical grade polycarbonate (PC) having an outer circumference 908 and an active area 904 having an inner circumference 906. An alignment marking 910 is displayed between the outer circumference 908 and inner circumference 906, which assists in the proper alignment and seating of the lens 402 in the laser device. In one embodiment, the lens 402 is 0.6 millimeters, and configured for a laser emitting a collimated single edge beam with a wavelength of 532 nanometers. In one embodiment, the active area 904 of the lens has a diffuser pattern structure providing, when used in conjunction with a laser having the aforementioned properties, displays the staircase pattern depicted in FIG. 7 and FIG. 8. As described above, the lens having a diffractive optical element for use in displaying a visual gait stimulation cue such as a descending staircase, may be integrated into the laser device of the gait monitoring and stimulation device described and depicted herein.

Referring now to FIG. 10, illustrating a block diagram showing exemplary components of an alternate embodiment of a gait monitoring and stimulation device. In one embodiment, the gait monitoring and stimulation device 1000 can be embodied in a mobile computing device including system storage 1024, memory interface 1022, central processor unit(s) 1026, input/output (“I/O”) and peripheral devices interface 1002. Sensors, devices, and subsystems can be coupled to an I/O and peripheral device interface 1002 to facilitate multiple functionalities. For example, one or more cameras 1007, accelerometers 1004, laser(s) 1005, display(s) 1006, global positioning system (“GPS”) transceiver 1008, communications subsystem 1010, and audio subsystem 1012 can be connected to I/O and peripheral devices interface 1002, in communication with the processor, to aid in driving various functions of the device 1000. For example, in some embodiments, the GPS transceiver 1008, in communication with the processor, may send/communicate geographic data (such as map coordinates) to said processor, which may utilize such geographic data to locate the position of the mobile computer processing device and determine whether the device is in motion. In one embodiment, the processor determines the distance traveled by the device over a predetermined period of time by utilizing geographic data communicated from said global positioning system. If the processor determines that the distance traveled by the device over the predetermined period of time does not exceed a predetermined distance, said processor activates said laser device. If the device is found not to have traveled in excess of such predetermined distance over the course of the predetermined period of time, the gait monitoring and stimulation device may be configured to deem such non-movement as an indication of a gait irregularity event, leading to the activation of one or more gait stimulation cues discussed herein, such as the laser device, audio playback of auditory cues, and/or activation of the vibrating motor. In one embodiment, a display 1006 implemented in the gait monitoring and stimulation device may be utilized to facilitate the display of, among other items, a graphical user interface (or “data interface”) for selecting one or more gait stimulation cues by either a user and/or a user's physician. For example, in one embodiment, a user may utilize a GUI on on a touchscreen display to select certain a laser-displayed visual cue from one of many provided, and/or one or more audio cues that he or she has found to be effective in alleviating gait freezing events. In one embodiment, the display 1006 may utilize various technologies such as LCD, Oxide LCD, a-Si, and TFT LCD display technologies to depict text and other information graphics in a high-resolution rendering.

In one embodiment of the gait monitoring and stimulation device, or more accelerometers may be utilized as sensors for the detection of movement by the user indicating a gait irregularity event (freezing event) which, per the steps discussed herein, may lead the device to activate one or more gait stimulation cues. In other embodiments, one or more accelerometers of the gait monitoring and stimulation device, in communication with the processor, may be utilized to detect the orientation or “tilt” of the gait monitoring and stimulation device to enhance the safety of use of the device. For example, in one embodiment of the gait monitoring and stimulation device, a 3-axis accelerometer may be utilized to determine whether the gait monitoring and stimulation device is being held in an orientation that is in excess of sixty degrees above a horizontal orientation. If the one or more accelerometers detect that the orientation or tilt of the gait monitoring and stimulation device exceeds sixty degrees angle above a horizontal orientation, a signal is transmitted to the processor, which determines the orientation of the device from data communicated from the accelerometers, results in the processor deactivating any visual stimulation cues and more specifically, the deactivation of the laser device that, because of the device orientation, otherwise cause damage to the eyes of a user or other adjacent person or service animal. It is contemplated that other threshold orientations/angles, other than the aforementioned sixty-degree angle above horizontal orientation mentioned above, can be implemented in other embodiments of the gait monitoring and stimulation device. Likewise, in alternate embodiments of the device, gyroscopic sensors may be used, alone or in combination with one or more accelerometers, to provide the device with orientation/tilt position data needed to determine whether the device is tilted upwards more than a predetermined threshold angle. The foregoing use of orientation/tilt sensors in connection with gait stimulation devices provides an advantage over prior art gait stimulation devices in that it enhances the safety of users and other persons in the vicinity of the device because it prevents the inadvertent shining of the laser beam into the eyes.

Functions related to communications can be facilitated through one or more communication subsystems 1010 that can include one or more wireless or wired communication subsystems. Wireless communication subsystems can include radio frequency receivers and transmitters 1011, and/or optical (e.g., infrared) receivers and transmitters. Wired communication systems can include a port device, e.g., a Universal Serial Bus (USB) port or some other wired port connection that can be used to establish a wired connection to other computing devices. In one embodiment of the gait monitoring and stimulation device 1000 embodying aspects described herein, an audio subsystem 1012 can be coupled to a speaker 1013 and one or more microphones 1014 to provide voice-enabled functions, such as voice recognition, voice replication, digital recording, and telephony functions.

For example, in one embodiment, a microphone may be utilized to facilitate voice-activation/deactivation of one or more gait stimulation cues, or to activate/deactivate gait monitoring, such that it is not necessary for the user to utilize his or her hands to activate/deactivate the device. In one embodiment, a microphone in communication with a processor of the gait monitoring and stimulation device samples audio input received via the microphone, the processor determines a volume of sound received over a predetermined period of time by utilizing audio data communicated from said microphone, wherein if said processor determines that the volume of sound received by said microphone over said predetermined period of time does not exceed a predetermined volume of sound threshold, said processor activates the laser or other stimulation cue/mode discussed herein.

Such functionality may also provide for a friend, family member, or caregiver of the user to activate the stimulation cues of the gait monitoring and stimulation device when such person notices that the user may be experiencing a gait freezing event. The use of “wake words” or spoken triggers, to interact with the device and to issue verbal commands that may be recognized by the device, is described in further detail below. The microphone 1014 may also be utilized to sense sound waves in the vicinity of the device such that the absence of soundwaves, or such that the volume of any such sound waves does not exceed a predetermined sound volume threshold, may indicate that the user is experiencing a gait freezing event that may result in the activation of one or more gait stimulation cues as described herein.

In another example, the communications subsystem may be utilized to transmit alerts indicating gait freezing events to non-users of the gait monitoring and stimulation device. In one embodiment, if the gait monitoring and stimulation device receives sensor data (GPS, accelerometer, audio) that indicate that the user of the gait monitoring and stimulation device is experiencing a freezing event, the device may be configured to transmit an alert to a predetermined non-user contact of the user via a cellular, Wi-Fi, or other network. In one embodiment, such an alert may communicate, via a text message, the identity of the user possibly experiencing a freezing event, and also geographical data such as geographical coordinates of the device (corresponding to the geographical location of the user), utilizing the GPS transceiver to determine such geographic location. The gait monitoring and stimulation device may be configured to provide a user and/or physician to select one or more contacts to receive such an alert Likewise, the gait monitoring and stimulation device may be configured to transmit such alerts to emergency responder dispatch (for example, an EMS dispatch service) services. In one embodiment, the gait monitoring and stimulation device may be configured to utilize a speaker 1013 to provide an announcement, capable of being heard by persons adjacent to the user, that the user is experiencing a freezing an event and providing further instructions that may facilitate assistance being offered to such user.

Input/control devices 1016 can include a touch controller and a touch surface 1018, and/or other input controller(s) such as a keyboard and/or mouse 1020. The touch controller can be coupled to the touch surface on the display 1006 for directing and processing signals from the touch surface to the processor. A touch surface and touch controller 1018 can, for example, detect contact and movement using any of a number of touch sensitivity technologies, including but not limited to capacitive and resistive technologies, as well as other proximity sensor arrays or other elements for ascertaining one or more points of contact with the touch surface. In one implementation, a touch surface can display a virtual keyboard 1020, which can be used as an input/output device by the user. Other input controller(s) can be coupled to other input/control devices, such as one or more buttons, rocker switches, thumb-wheel, infrared port, USB port, and/or a pointer device such as a stylus (not shown).

In embodiments of the gait monitoring and stimulation device, a memory interface 1022 can be coupled to system storage 1024 and central processor unit(s) 1026. System storage 1024 may include volatile high-speed random-access memory 1028 or non-volatile memory 1030. In one embodiment of the mobile computer processing device, the system storage may include storage media technologies such as RAM, ROM, EEPROM, flash memory or other memory technology, or any other medium which can be used to store desired information, and which can be accessed by the device.

The storage system may also store instructions to facilitate the operation of the gait monitoring and stimulation device, and communications with one or more additional computing devices, such as one or more computing devices comprising embodiments of the gait monitoring and stimulation device. Operating system instructions 1032 for the computer processing device may be stored in the storage system. Operating system software such as iOS, Android, Darwin, RTXC, LINUX, UNIX, OS X, or WINDOWS may be used to facilitate operation of the device. For example, operating system instructions may include instructions for handling basic system services and for performing hardware dependent tasks. One or more central processor units 1026 are connected to the memory interface 1022, which is in turn connected to the storage system. Such processor(s) may run or execute the operating system and various other software programs and/or sets of instructions stored in memory to perform various functions for the gait monitoring and stimulation device.

The storage system may include graphical user interface instructions 1034 to facilitate graphic user interface processing to facilitate web browsing-related processes and functions and display GUIs for facilitating communications to and from the device; and instructions for a gait monitoring and stimulation device application 1038 that is capable of displaying GUIs and providing other functionality of the gait monitoring and stimulation device as described herein. The storage system memory may also store other software instructions for facilitating other processes, features and applications, such as applications related gait monitoring and gait stimulation.

In an embodiment of the gait monitoring and stimulation device, the storage system may include one or more storage databases 1031 stored preferably in non-volatile memory 1030. Such databases may store information such as software, data associated with gait monitoring and gait stimulation, audio files for use in providing auditory stimulation cues, gait stimulation modes selected by a user, physician, or caregiver, other user information, drivers, and/or any other data item utilized by the gait monitoring and stimulation device taught herein.

In one embodiment, the gait monitoring and stimulation device further includes a power control unit and one or more batteries 1044. The power control unit 1044 is configured to control the amount of power consumed by the device. Those of skill in the art will recognize that by actively controlling the amount of power consumed by the device, the device may achieve more efficient use of electrical energy that is consumed by the device. The power control unit may include a clock and/or timer for precise control of power consumed by the gait monitoring and stimulation device. The power control unit may include any combination of hardware and software, and digital and/or analog circuitry. The power control unit (also may be referred to or further include a battery management unit) may include one or more microcontrollers and/or other hardware modules. Embodiments of the gait monitoring and stimulation device may include one or more rechargeable batteries or other battery system for powering the device, including one or more batteries coupled together in parallel or series configuration to output any desired voltage and/or current. One or more batteries may be implemented by utilizing rechargeable battery chemistry including, but not limited to, nickel metal hydride (NiMH), lithium polymer, and lithium ion battery chemistries. Embodiments of the gait monitoring and stimulation device may include solar cells for the recharging of batteries.

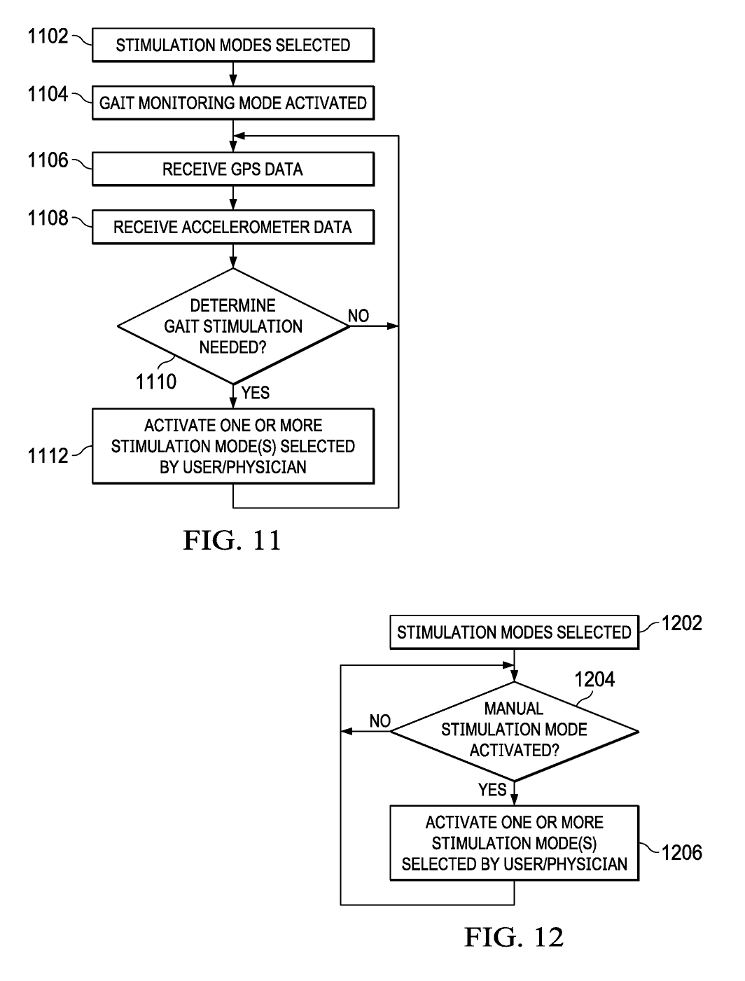

Referring now to FIG. 11, a process flow diagram illustrating steps of monitoring for gait irregularities and providing gait stimulation cues performed by an embodiment of the gait monitoring and stimulation device is shown. In one embodiment, multiple gait stimulation types or “stimulation modes” may be provided to a user for the purpose of causing the cessation of a gait freezing event or to reduce the likelihood of such a gait freezing event occurs. One type of gait stimulation mode or cue is the laser projection of a pattern via a laser device having a lens with a diffractive optical element of the type described with reference to FIGS. 7-9. Other types of gait stimulation modes may include auditory stimulation modes or cues. For example, one or more auditory cues may be utilized alone, or in combination with visual cues provided by visual stimulation modes, to cause the cessation of gait freezing events in users with Parkinson's disease or other neurological disorders.

In one embodiment of the gait monitoring and stimulation device, the audio subsystem as depicted in FIG. 10, may be utilized to play prerecorded auditory cues to a user via a speaker or transmitted to wireless headphones/ear pieces (for example, via Bluetooth protocols). For example, in one embodiment, audio data recorded from a person walking along a path filled with gravel or other action-relevant sounds, may be stored in a storage database in non-volatile memory as described in connection with FIG. 10. Such audio data may be played, in a loop, via the audio system as a stimulation mode selected by the user or by a user's physician or caregiver. Such audio data may be stored in the storage database. For example, in one embodiment, the processor may determine that the distance traveled by said device over a predetermined period of time, utilizing GPS geographical data, does not exceed a predetermined distance, resulting in said processor activating the playback, via wireless headphones in communication with said device and worn by a user of the device, of said audio data comprising a sound recording of, for example, a person walking on gravel. In other alternate embodiments, the gait monitoring and stimulation device may include a vibrating motor controlled by the processor, to provide vibration to the user as a stimulation mode. The specific stimulation modes may be selected 1102 by the user/physician/caregiver using a graphical user interface and inputted by such persons by a touchscreen interface as discussed in connection with the embodiment of the device as shown in FIG. 10.

Still referring to FIG. 11, the gait monitoring mode is activated by, for example, the touchscreen display interface described above. In alternative embodiments, the gait monitoring and stimulation device may utilize audio monitoring for certain spoken triggers or “wake words,” allowing the user to announce audio commands to the device via the microphone (see FIG. 10 at 1014), thereby activating the gait monitoring mode. In one embodiment, the device may receive and analyze global positioning system (GPS) geographical data 1106 from the GPS transceiver, determining that gait irregularity event is occurring should such GPS data indicate non-movement or decreased movement of the device during a predetermined sampling period. Likewise, the device may be configured to receive accelerometer data 1108 from one or more accelerometers integrated within the device, and determine that a gait freezing event is occurring should such accelerometer sensors detect little to no movement of the device during a predetermined event. In other embodiments, the microphone(s) of the device may be utilized to provide for gait monitoring, determining that a gait freezing event is occurring should the microphones sense sound waves below a predetermined threshold volume level over a predetermined sampling period of time.

Such data, which may include GPS, accelerometer, and auditory sensor data, is analyzed by the processor of the device, and a determination 1110 is made as to whether a gait freezing event is probable, due to one or more types of sensor data falling below predetermined thresholds, such that the activation of the one or more gait stimulation modes is needed. As discussed above, sensor data falling below a predetermined threshold may result in a determination that the activation of gait stimulation mode(s) is needed. For example, if the GPS data received indicates that the device has not moved more than ten feet in a thirty-second sampling period, the gait stimulation device instructions (see FIG. 10 at 1038) may deem that a gait freezing event is likely occurring and will activate 1112, via the processor, one or more gait stimulation modes or cues (visual, audio, or vibratory cues). It is contemplated that such visual/audio/vibratory cues may be activated alone, or in combination. It is also contemplated that user, physicians, and caregivers may modify the predetermined threshold levels of movement or sound, which would cause the device to determine that a gait freezing event is occurring, and that activation of one or more gait stimulation modes is needed. It is also contemplated that a user, physician, or caregiver may modify, using a GUI interface, the types of sensor data that may be utilized to determine a gait freezing event. As depicted in FIG. 11, selected stimulation modes, once activated, will continue to operate until gait stimulation is deemed no longer necessary in response to the one or more sensor data received by the device.

Referring now to FIG. 12, a process flow diagram illustrating steps for the manual activation of selected gait stimulation modes performed by an embodiment of the gait monitoring and stimulation device is shown. As described in connection with FIG. 11 above, one or more gait stimulation modes or “cues” may be selected 1202 by a user/physician/caregiver for use in connection with the gait monitoring and stimulation device. The user, via a user interface, may be allowed to set 1204 the device to provide for the manual activation of one or more gait stimulation modes. In one embodiment, rather than actively monitoring for gait freezing events as described in connection with the process of FIG. 11, a user or other person may be given the ability to manually activate 1206 one or more gait stimulation modes or cues. For example, in one embodiment, a button may be positioned on the gait monitoring and stimulation device (for example, at FIG. 1 at 110) to allow the user to activate a visual stimulation, audio stimulation, or vibratory stimulation mode. As described above, the device may be configured to receive verbal commands via a microphone through use of a spoken trigger or wake word, such commands being used to manually activate 1206 one or more gait stimulation modes. For example, a caregiver of a user may notice that a gait freezing event is occurring or is likely about to occur to the user of the device. Such caregiver may issue a verbal command to the device, utilizing a wake word followed by a command, thereby activating one or more gait stimulation modes. Likewise, the wake word followed by a verbal command, may deactivate the one or more gait stimulation modes of the device. For example, U.S. Pat. No. 9,886,953, issued on Feb. 6, 2018, incorporated by reference herein, describes the sampling of audio input received via a microphone, determining whether the audio input comprises a spoken trigger or wake word, and in response to a spoken trigger, activating a virtual assistance session which may be utilized to provide for the activation and deactivation of gait stimulation modes. As depicted in FIG. 12, the gait stimulation mode(s) will remain active until deactivated by a user or other person.

The invention may be embodied in other specific forms without departing from the spirit or essential characteristics thereof. The present embodiments are therefore to be considered in all respects as illustrative and not restrictive. Accordingly, the scope of the invention is established by the appended claims rather than by the foregoing description. All changes that come within the meaning and range of equivalency of the claims are embraced therein. Further, the recitation of method steps does not denote a particular sequence for execution of the steps. Such method steps may therefore be performed in a sequence other than that recited unless the particular claim expressly states otherwise.

I claim:

1. A gait monitoring and stimulation device for comprising:
a housing having an anterior end and a posterior end;
a laser device having a lens with a diffractive optical element configured, such that when said laser device is activated, a laser beam passes through said lens and said laser device projects a two-dimensional laser image, said laser device being mounted within said housing such that at least a forward portion of said laser device protrudes from said housing;
a processor mounted within said housing; and
one or more accelerometers in communication with said processor,
wherein said processor determines the orientation of said device by utilizing orientation data communicated from the one or more accelerometers,
wherein if said processor determines that the orientation of the device exceeds a predetermined threshold angle with respect to a horizontal orientation, said processor deactivates said laser device.

2. The gait monitoring and stimulation device of claim 1, wherein said two-dimensional image comprises a plurality of adjoining trapezoids.

3. The gait monitoring and stimulation device of claim 1, wherein said laser device further comprises a lens tip body removably mounted on said forward portion of said laser device.

4. The gait monitoring and stimulation device of claim 3, wherein said lens is mounted within said lens tip body.

5. The gait monitoring and stimulation device of claim 1, further comprising a global positioning system transceiver in communication with said processor,
wherein said processor determines the distance traveled by said device over a predetermined period of time by utilizing geographic data communicated from said global positioning system.

6. The gait monitoring and stimulation device of claim 5, wherein if said processor determines that the distance traveled by said device over said predetermined period of time does not exceed a predetermined distance, said processor activates said laser device.

7. The gait monitoring and stimulation device of claim 1, further comprising a microphone in communication with said processor,
wherein said processor samples audio input received via the microphone,
wherein said processor determines a volume of sound received over a predetermined period of time by utilizing audio data communicated from said microphone,
wherein if said processor determines that the volume of sound received by said microphone over said predetermined period of time does not exceed a predetermined volume of sound threshold, said processor activates said laser device.

8. The gait monitoring and stimulation device of claim 1, further comprising a storage database in communication with said processor, said storage database including audio data.

9. The gait monitoring and stimulation device of claim 8, further comprising a global positioning system transceiver in communication with said processor,
wherein said processor determines the distance traveled by said device over a predetermined period of time by utilizing geographic data communicated from said global positioning system,
wherein if said processor determines that the distance traveled by said device over said predetermined period of time does not exceed a predetermined distance, said processor activates the playback, via wireless headphones in communication with said device, of said audio data.

10. The gait monitoring and stimulation device of claim 1, further comprising a microphone in communication with said processor,
wherein said processor samples audio input received via the microphone,
wherein said processor determines whether the audio input comprises a spoken trigger, and in accordance with a determination that said audio input comprises the spoken trigger, activate a virtual assistant session.
